(19) United States
(12) Patent Application Publication
(10) Pub. No.: US 2024/0242843 A1
(43) Pub. Date: Jul. 18, 2024

(54) REDUCING ADVERSE HEALTH EVENTS IN ASSISTED LIVING

(71) Applicant: NOVA SOUTHEASTERN UNIVERSITY, Fort Lauderdale, FL (US)

(72) Inventors: Johannes Vieweg, Fort Lauderdale, FL (US); Julie A. Jacko, Fort Lauderdale, FL (US); François Sainfort, Fort Lauderdale, FL (US)

(73) Assignee: NOVA SOUTHEASTERN UNIVERSITY, Fort Lauderdale, FL (US)

(21) Appl. No.: 18/561,778

(22) PCT Filed: May 18, 2022
(86) PCT No.: PCT/US22/29757

§ 371 Date: Nov. 17, 2023

Related U.S. Application Data
(60) Provisional application No. 63/189,816, filed on May 18, 2021.

(51) Int. Cl. G16H 50/30; G16H 40/20; G16H 40/60; G16H 50/70

(52) U.S. Cl. CPC G16H 50/30; G16H 40/20; G16H 40/60; G16H 50/70

(57) ABSTRACT

A computer system and a computer-implemented method reduce adverse health events for at-risk resident patients. The method collects a resident's objective health data using the resident's health history and current evaluation of the resident's cognitive abilities, functional abilities, and comorbidities. The method analyzes the collected objective health data to determine if a resident requires assistance and assigns the resident either: (a) independent living to a resident with normal cognitive function and no functional disabilities, (b) assisted living to a resident unable to carry out activities of daily living, and (c) memory care living to a resident with a dementia diagnosis. If either (b) or (c) is assigned, the method provides at least one of wearable sensors on the resident and sensors within the living environment of the resident, which such sensors provide sensor data that is analyzed to determine if the resident requires assistance.

### BACKGROUND

This disclosure relates to preventing adverse health events for residents of assisted living, and more particularly to optimizing a selection of a residence type and associated sensors to minimize the frequency of, and quickly respond to, adverse health events.

Many assisted living residents need help with activities of daily living. For example, they made need help with bathing (67%), walking (57%), dressing (52%), toileting (45%), bed transfer (31%), and eating (18%). Of the assisted living residents in Florida, 47% are over the age of 85, and 46% have Alzheimer's disease or another form of dementia. Nationwide, assisted living residents include 71% females and 29% males, with 52% at least 85 years of age, and 30% between 75 and 84.

Currently, resident selection criteria for an assisted living facility are typically based on individual or familial preference. The decision can be guided by imprecise clinical metrics or a perceived health state. This leads to increased dissatisfaction among residents, and can result in a lack of quality care and a decreased quality of life.

In 2017 the FDA announced a transformation of its regulatory practice for approving and certifying medical devices, including the Digital Health Software Precertification (Precert) Program. The Precert program is part of a new regulatory model to assess smart phone apps, wearables, sensors, and software for health/healthcare applications. The Precert program would affect devices such as the FITBIT or APPLE WATCH devices, as well as other devices used to monitor health.

### BRIEF SUMMARY

A computer-implemented system for reducing adverse health effects for at-risk patients, comprises: one or more computing devices executing instructions, stored on media readable by the one or more computing devices, to: store data pertaining to historical physical manifestations of various health problems in a set of at-risk people: store data pertaining to at-risk patients including health problems of each patient: for each patient, select a plurality of electronic monitoring devices which each detect at least one of the physical manifestations of a health problem of the patient and report the selection of devices whereby the devices can be positioned upon at least one of the patient or an environment in which the patient lives: for each patient, based on the electronic monitoring devices that have been positioned, electronically monitor the devices to determine if one or more manifestations of a health problem of the patient has taken place: use the determination to evaluate whether the patient is at a predetermined higher probability of an adverse health effect relative to a baseline probability for the patient, and report, by sending an alert message to a selected computer device associated with a medical or health professional, the higher probability whereby assistance can be directed to the patient; and add data pertaining to the predetermined higher probability to the data pertaining to historical physical manifestations.

A computer-implemented method reduces adverse health events for at-risk patients. The method comprises: collecting, with a processor accessing records in a computer database, objective health data of at least one individual resident patient (resident) using a health history of the resident, which is stored in one or more records in the computer database, and objective health data collected from a current evaluation of cognitive abilities, functional abilities, and comorbidities, of the resident: using the collected objective health data to assign (a) independent living to a resident with normal cognitive function and no functional disabilities, (b) assisted living to a resident unable to carry out a plurality of activities of daily living, and (c) memory care living to a resident with a dementia diagnosis: if the collected objective health data of a resident are used to assign (b) or (c), provide at least one of wearable sensors on the resident and sensors within the living environment of the resident, which provide sensor data pertaining to a real time health state of the resident living within the assisted living or memory care environment, the type of sensor selected being based upon the collected objective health data; communicate the sensor data in real time to at least one of a health expert and a computer algorithm to analyze the sensor data to determine if the resident requires assistance; and reevaluate a correlation of the collected objective health data, selection of sensors, and analysis of sensor data for a plurality of patients to reduce a requirement of assistance of patients.

### BRIEF DESCRIPTION OF THE DRAWINGS

The accompanying figures in which like reference numerals refer to identical or functionally similar elements throughout the separate views, and which together with the detailed description below are incorporated in and form part of the specification, serve to further illustrate various embodiments and to explain various principles and advantages all in accordance with the present disclosure, in which:

FIG. 1 depicts a phenotyping conceptual model in accordance with the disclosure;

FIG. 2 depicts a projection of individuals and ages in need of assisted living support;

FIG. 3 depicts selection among categories of living based upon phenotyping of the disclosure;

FIG. 4 depicts a contact center organization helpful for carrying out the disclosure;

FIG. 5 depicts a flow of data from wearable/environmental sensors resulting in intervention as needed, in accordance with the disclosure;

FIG. 6 depicts an overall infrastructure for carrying out the disclosure;

FIG. 7 depicts additional detail of a data architecture for carrying out the disclosure;

FIG. 8 depicts information technology details for maintaining security and patient privacy when carrying out the disclosure;

FIG. 9 depicts mathematical formulations used in FIGS. 10-11;

FIG. 10 depicts mathematical models for phenotyping, living category assignment, and sensor assignment;

FIG. 11 depicts a mathematical model in accordance with the disclosure for predicting a health condition of a resident;

FIG. 12 depicts relationships of actors associated with carrying out the disclosure;

FIG. 13 depicts pathways for gathering, processing, and acting upon data in accordance with the disclosure;

FIG. 14 depicts various components of an embodiment of a system according to the disclosure for monitoring patients for observable manifestations relating to potential adverse health events;

FIGS. 15 and 16 are a PRIOR ART depiction of a graphical representation of common diseases leading to death in people at least 70 years old (https://vizhub.healthdata.org/gbd-compare/, Institute for Health Metrics and Evaluation, University of Washington), which data can be included in a decision regarding a selection of diseases to monitor using an example system according to the disclosure. Some examples of certain health problems that can be ameliorated through a change in behavior are identified by encircled letters A-D; and

FIGS. 17, 18, and 19 are examples of graphical data representations on one or more displayed user interface(s) as shown in FIG. 14 with reference to Component 7, according to various embodiments.

### DETAILED DESCRIPTION

This written description uses examples to disclose the embodiments, including the best mode, and also to enable those of ordinary skill in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. Note that not all of the activities described above in the general description or the examples are required, that a portion of a specific activity may not be required, and that one or more further activities can be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below: Accordingly, the specification and figures are to be regarded in an illustrative rather than a restrictive sense, and all such modifications are intended to be included within the scope of invention.

It can be advantageous to set forth definitions of certain words and phrases used throughout this patent document. The term “communicate,” as well as derivatives thereof, encompasses both direct and indirect communication. The term “discreet,” as well as derivatives thereof, references to the amount of skin exposed by a user of the garment, rather than the type of style of the garment. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A: B: C: A and B: A and C: B and C; and A, B, and C.

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the specification, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

Headings are provided for the convenience of the reader, and are not intended to be limiting in any way.

The inventors have recognized a need for advancement and direct adherence to individual comorbidities of assisted living residents, other senior living environments, and the environments of other at risk people (herein, collectively “residents”). The instant disclosure present methods, systems and configurations to create a senior living “built environment” that enhances the longevity and quality of life of older adults. The disclosure may be considered to present three pillars to address this: phenotyping, a clinical contact/call center, and predictive modeling. Together, these three pillars form a closed loop system that does not currently exist in the senior living industry.

In accordance with the disclosure, wearable and fixed monitoring technologies are utilized for biometric, behavioral, and activity tracking, and are selected in accordance with a resident's “phenotype”, which is considered herein to including health risks and/or the presentation of a condition/disease. These data are then translated into real-time health coordination, intervention and disease prevention strategies that are coordinated through a multiregional data architecture, collectively creating a closed loop system in which the actionable health data are immediately translated into appropriate clinical interventions or changes/adjustments to the resident's built environment.

The three pillars can more particularly include any or all of the following:
methods, systems, and configurations to create senior living environments utilizing health data through customized monitoring technologies;
methods for phenotyping and selection of older adults for a best fit to residential configuration (Assisted/Independent/Memory Care facility) and monitoring technologies given health risks and/or the presentation of a disease in a given individual;
methods and systems to connect a monitored senior living environment with other senior living or health facilities (regional or remote);
methods and systems to utilize and analyze data acquired through biosensor and monitoring technologies for health coordination, health/built environment interventions, and disease prevention, that can be processed through a clinical call center;
methods and systems to build multiregional data architecture for biologic sensors and monitoring technologies in the built environment;
methods and systems for biosensor and monitoring technology evaluation, customization, and integration into the built environment for older adults:

methods and systems for continuously tracking the locations of individuals within a senior living built environment using smart Bluetooth, low energy networks and/or indoor GPS systems; and methods and systems for producing predictive health models, including artificial intelligence algorithms.

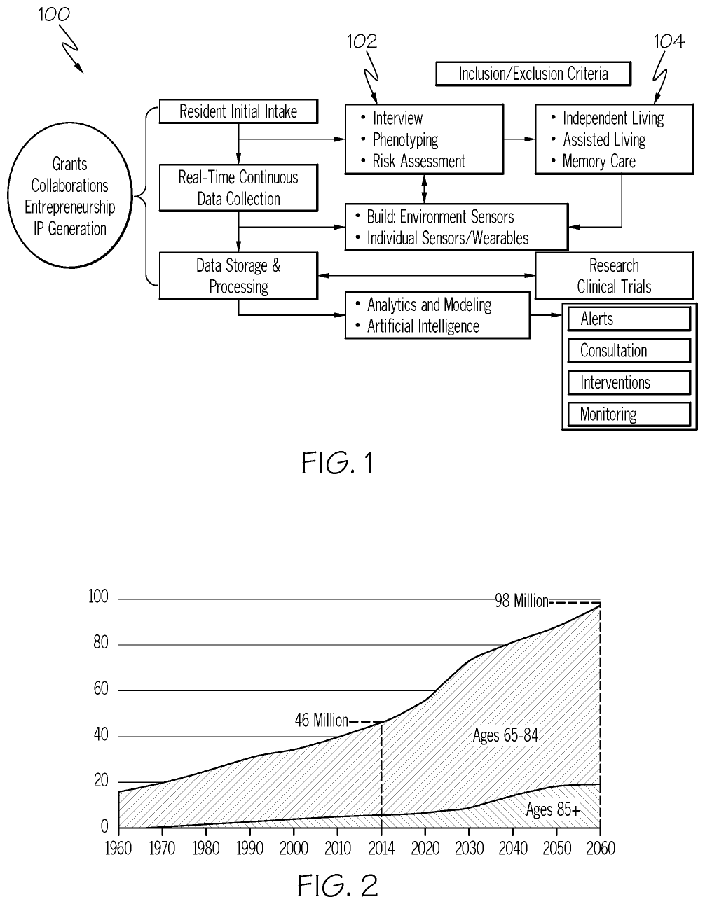

With reference to FIG. 1, the disclosure provides customized monitoring technologies which are utilized in the built environment to enhance the longevity and quality of life of older adults. Technologies are selected in accordance with health risks, acute conditions, chronic conditions and/or the presentation of disease in a given individual. Health data is obtained through customized sensor and monitoring technologies and translated into real-time health coordination, interventions, and disease prevention through a multiregional data architecture.

With reference to FIG. 1, an operational and functional system 100 of the disclosure entails a three-step, sequential process of: 1) older adult phenotyping and selection: 2) real-time data collection through bio-activity, and behavioral sensors transmitted to a clinical contact/call center across health networks; and 3) predictive modeling of future health states and adverse events that yield operational and health interventions across networks, that prevent adverse events and enhance longevity and health related quality of life of older adults in the built environment.

### Phenotyping and Selection of Older Adults

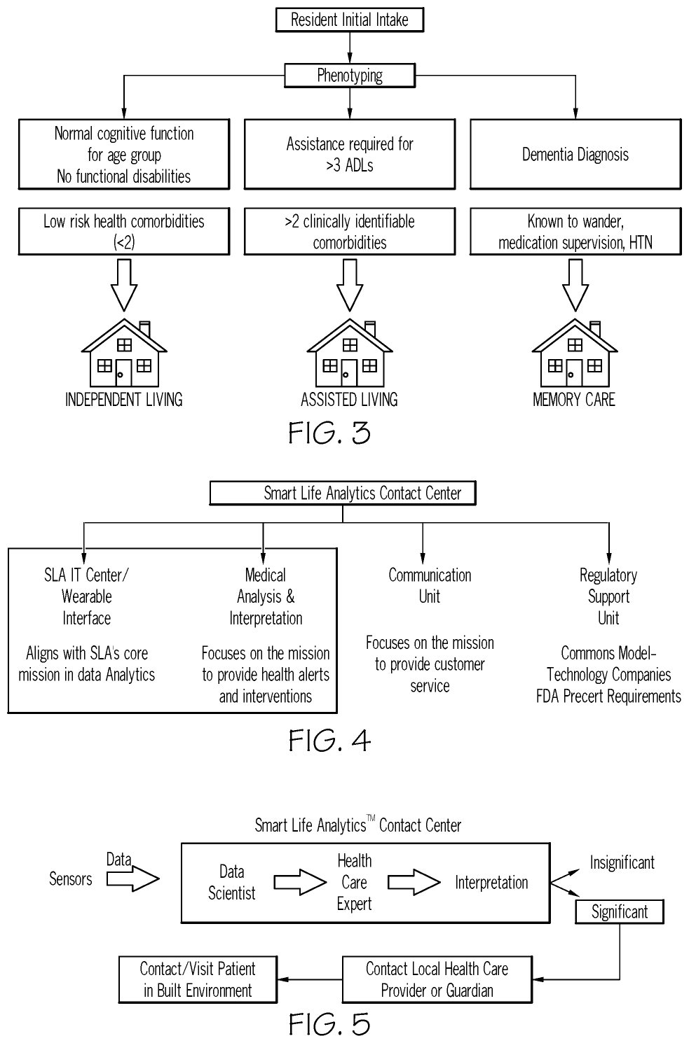

With reference to FIG. 3, older adults are selected for an appropriate senior living configuration based on phenotyping, obtaining health-related information, quality of life questionnaires, and performing risk assessments (FIG. 1, 102). As can be seen in FIG. 2, the population of residents, and particularly residents older than 85, is growing rapidly. The system 100 will triage seniors according to senior care living categories 104 (e.g., Independent Living, Assisted Living, or Memory Care). Older adults are matched to biosensor and built environment monitoring technologies according to phenotype, disease risk and pre-existing underlying conditions.

With further reference to FIG. 3, phenotyping of the disclosure is first carried by conducting an evidence based initial intake and comprehensive risk assessment. Evidence based includes using objectively gathered data, which reduces deliberate or unintentional personal bias. This can include, as examples, resident socio-demographics, such as age, gender, ethnicity, education level, income, location and other socio-demographic characteristics: medical history: health and injury risk factors; current vitals, lab tests, and physical exam results: physical function/abilities; social/behavioral function: cognitive function; comorbidities.

With reference to FIG. 4, a contact center includes direct support for an IT center/Wearable Interface Unit which aligns with a core mission in data analytics: a Medical analysis & interpretation Unit which focuses on the mission to provide health alerts and interventions: a Communication Unit which focuses on the mission to provide customer service; and a Regulatory Support Unit which coordinates a Commons Model and interfaces with various technology companies to meet FDA Precert requirements which pertain to the disclosure.

These factors are then applied to achieving three goals: (1) an optimal assignment among senior living choices, including independent living, assisted living, and memory care: (2) an optimal selection of sensors (worn and within the living environment) for each resident; and (3) an initial predictive risk stratification for a range of health and wellness conditions.

Phenotyping is a critically important first step, as it enables identifying the most clinically actionable digital health technologies: improves the quality of stay: minimizes adverse events. Additionally, phenotyping enables the development of weighted phenotypic markers for prospective medicine, using individual and collective phenotyping data. In a feedback or iterative process, application of phenotype data to the three goals is continuously improved.

### Data Coordination and Contact Call Center

Further in accordance with the disclosure, health, activity and behavioral data are obtained through customized monitoring technologies (sensors' herein), both wearable and embedded in the built environment (physical housing structure incorporating sensors), are transmitted to a centralized data coordinating facility (further detailed elsewhere herein) and analyzed in real time for adverse health risk or events. The data are translated in real-time into operational and health interventions that prevent disease and protect seniors from adverse health risks.

More particularly, and with reference to FIGS. 4 and 5, data from the sensors is communicated to a data system created by data scientists, where the system can apply filters or other processes to present the data in a more human readable and approachable form. The collected data is then presented to health care experts and/or to a computer analysis system, which can include sophisticated algorithms, such as an artificial intelligence (AI) system, to carry out an interpretation of the data. If the data are deemed significant from a health/risk perspective, relevant information corresponding to the data are forwarded to the resident's local health care provider or guardian, which can when needed result in a visit to the patient in the built environment for further analysis and follow up.

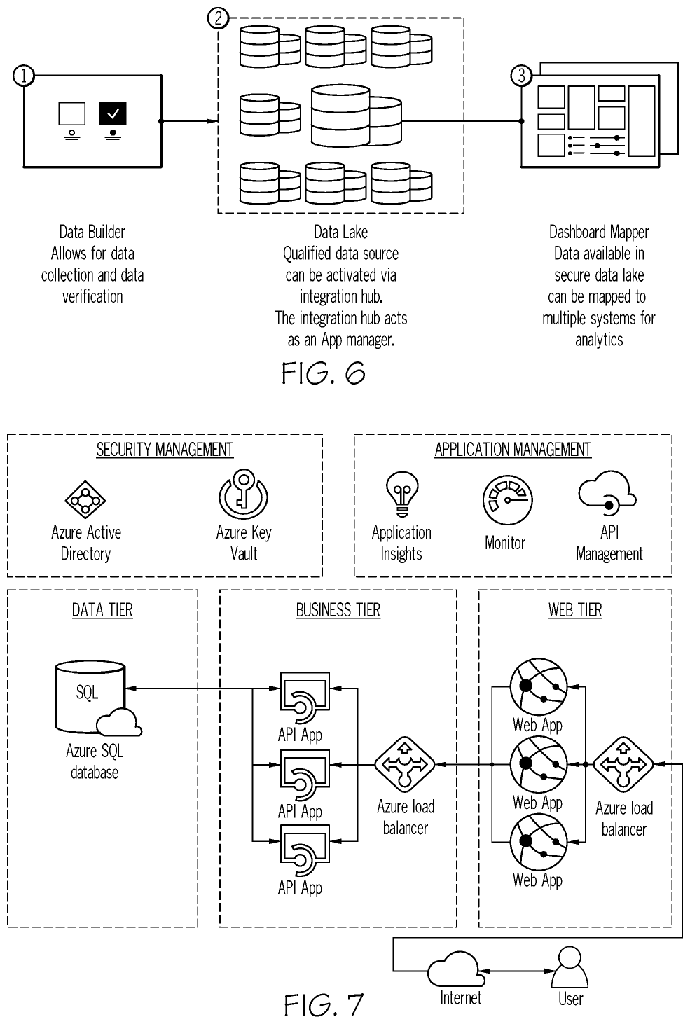

An overall infrastructure in support of this is shown in FIG. 6, a data architecture is shown in FIG. 7, and details regarding data security are shown in FIG. 8.

### Predictive Modeling

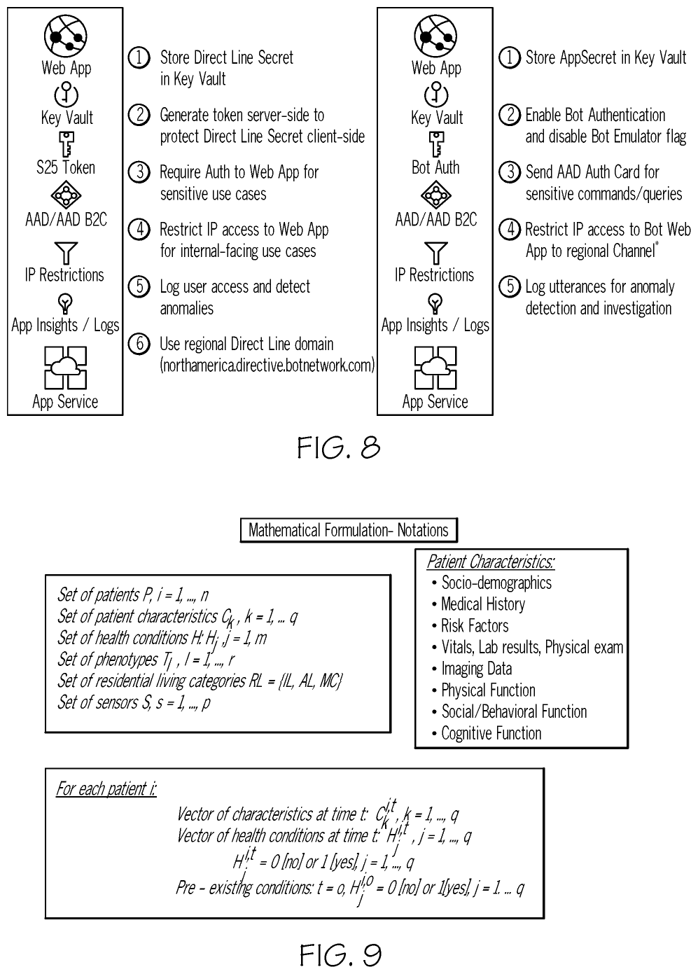

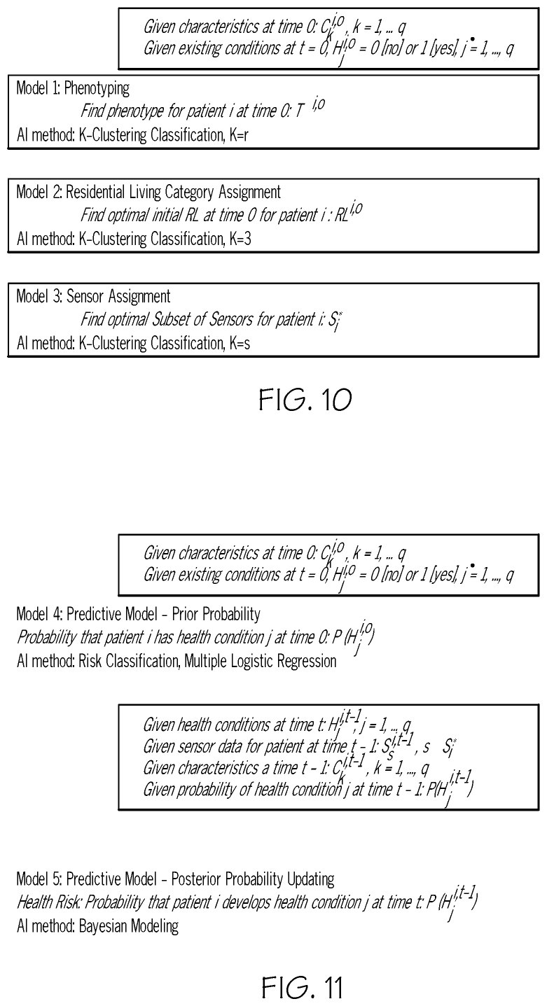

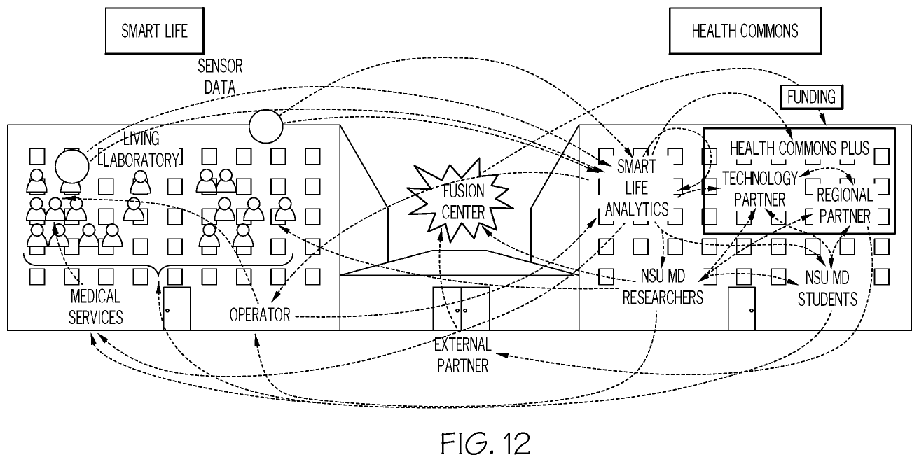

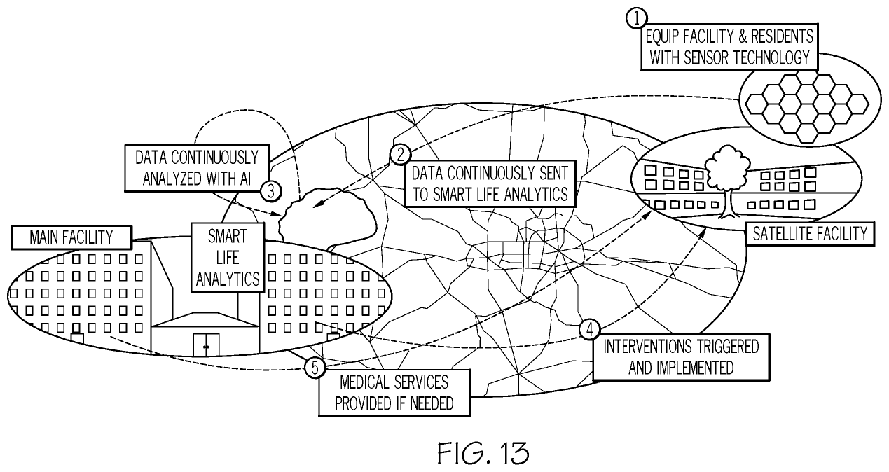

In accordance with the disclosure, in order to accurately phenotype, select and assign residents to the appropriate living category, assign the correct sensors, and produce accurate predictive models that yield actionable interventions, the data are subjected to mathematical algorithms for predictive analytics, as detailed in FIGS. 9-11.

### Scalability

A computer-implemented architecture according to the disclosure supports a centralized data coordinating facility comprised of three tiers: a Web Tier, a Business Tier, and a Data Tier. Security and Application Management would occur throughout the tiers. A user would access the application through the Internet and requests would pass through the three tiers for data entry or retrieval. In this manner, health data can be exchanged and operationalized across monitored senior living (and other health) environments using a distributed data architecture. Analytics including artificial intelligence are employed to generate prospective medical interventions for residents, reducing cost, and enhancing quality of life. FIG. 9 illustrates scalability of the system.

### Advantages of the Disclosure

The disclosure provides a proactive and predictive closed loop system to continuously improve results and quality of life for residents, and a feedback connection to organized clinical care and clinical experts. Further, the disclosure is applicable to all tiers of living: independent, assisted and memory care living configurations. Residents are phenotyped as detailed herein, and are therefore placed in an optimal living configuration based on the phenotype. Sensors are custom configured for specific diseases/conditions/risks for optimal match with individual phenotypes, which is optimized based on individual and collective phenotyping. Sensors are not limited to location/position/movement but also include significant bio and health sensors. Further, data are actively processed by one or more computer processing systems using sophisticated AI algorithms so that predictive models are produced that enable timely interventions that prevent adverse events and conditions.

According to various embodiments, as one example, the one or more computer processing systems comprise one or more processors communicatively coupled, such as via a bus architecture, with main memory, with persistent storage memory, with one or more network interface devices, and with one or more user interfaces. The one or more processors are communicatively coupled via the network interface devices with a database storage system. The database storage system contains records of medical and health-related information associated with individual resident patients. Additionally, according to the example, the one or more processors are communicatively coupled with a set of sensors—both wearable sensors tailored to individual health status/conditions as well as built-in sensors in a physical environment of individual resident patients.

The one or more processors operate the sophisticated AI algorithms so that the predictive models are produced and stored in the persistent storage memory and in the database storage system. These predictive models enable the computer processing systems to operate, according to the example, to generate and send information signals and alert signals to selected medical staff and professionals which can provide timely interventions to specific resident patients which prevent adverse events and conditions for those specific resident patients.

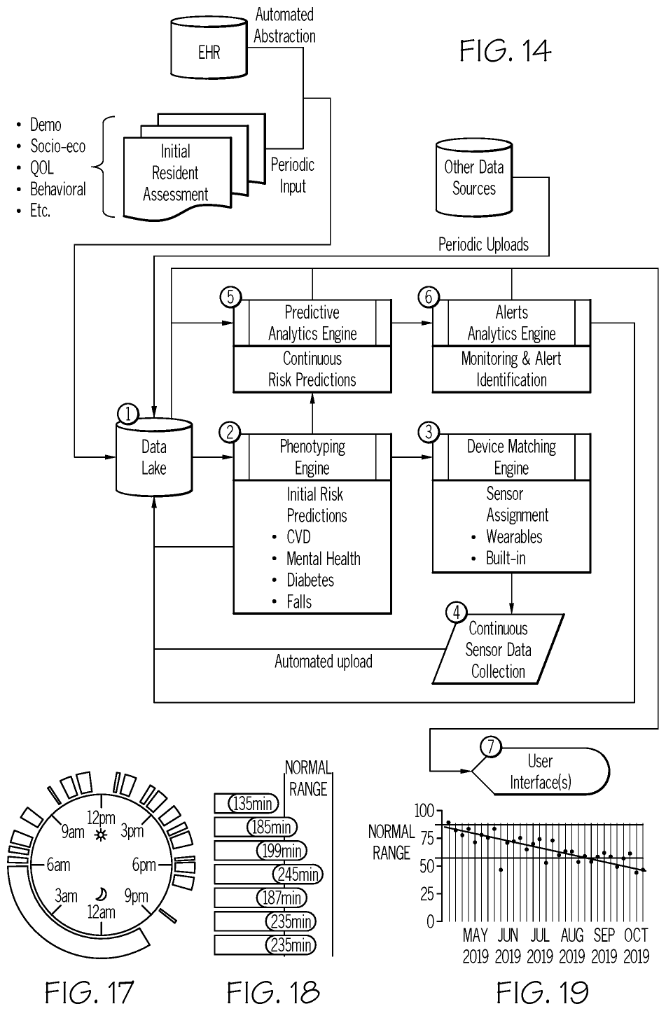

With reference to FIG. 14, according to various embodiments, the disclosure provides system 100, which for example comprises the one or more computer processing systems discussed above, to measurably enhance the health-related quality of life and lengthen the life span of older adults (>65 years) residing in senior living environments/facilities through 1) automated real-time monitoring of health status and healthy behaviors. 2) identifying potential adverse events before they happen, and 3) mitigating the occurrence of adverse events through timely 30) clinical and/or environmental interventions. FIG. 14 depicts an example of system 100, according to various embodiments, which can be considered an expert system as it analyzes medical conditions, assesses individual risks due to those conditions, selects electronic and other components which are used to measure the health and behavior of individuals as they relate to the conditions, and monitors and evaluates information from the components, and managing the flow of data relating to the individuals and their evaluations.

System 100, in the example, is comprised of a) an optimal set of sensors—both wearable sensors tailored to individual health status/conditions as well as built-in sensors in the physical environment: b) a data lake that securely and continuously stores and manages multiple streams of data; and c) a novel, artificial intelligence (AI)-driven, closed-loop expert system that comprises four AI engines: a phenotyping engine, a device matching engine, a predictive analytics engine and an alerts analytics engine.

System 100, continuing with the example, accurately assesses individual health risks in older adults and matches sensor or biometric technology to monitor for, and prevent, high-risk adverse events in the built environment. To benefit from system 100, seniors can elect (opt-in) to live in a custom-designed residential living facility equipped with unobtrusive sensor and digital data communication technology that enables both passive and active data collection about each senior resident's current health status, including how, when, and where senior residents perform daily activities within their residential spaces. The data is used to predict and prevent adverse health related events for residents/patients who are at-risk of an adverse health event due to a disease or other health condition.

Seven key components are identified in FIG. 14 as a number within a circle each representing a component.

Component 1: A data lake (repository) securely collects, stores, and manages multiple streams of data, in one or more physical locations. Initial resident assessment data are collected and entered into the data lake using data collection tools including, but not limited to demographics, socio-economic data, health-related quality of life assessments, initial comprehensive clinical examination, resident medical history, and full risk evaluation. In addition, automated data abstraction is performed from electronic health records (EHR) to gather clinical information which is needed or helpful to system 100. Finally, regional and national databases are queried for relevant data, and data are periodically uploaded to provide baseline and comparative population data.

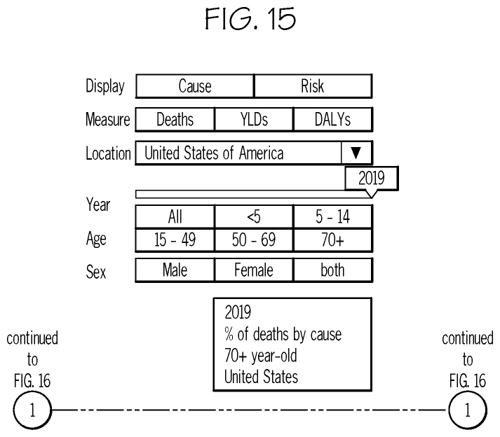

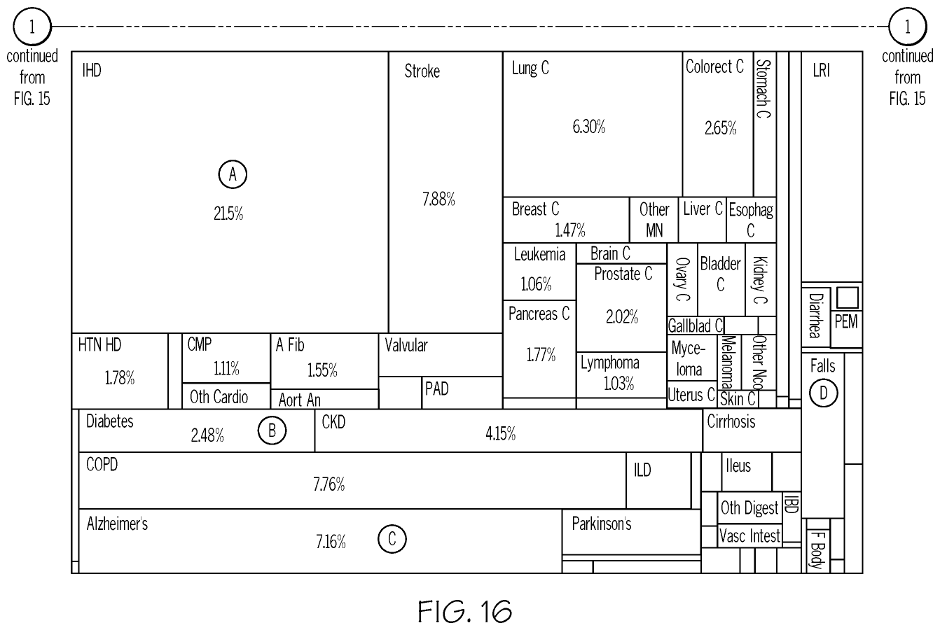

Component 2: When a resident is first enrolled within system 100, all initial data in the data lake are automatically accessed and analyzed by the first AI engine of the expert system, the Phenotyping Engine. This engine runs a series of AI algorithms and nomograms, stored on computer readable media, for example non-volatile media, by one or more electronic data processors, to phenotype the resident according to the disclosure, which includes multiple logistic regression and other risk classification methodologies. The result of the analysis is a full risk profile for the resident that estimates the risk (probability) with respect to at least four disease/condition phenotypes which can include for example cardiovascular disease, mental health, diabetes, and falls. These four conditions are advantageously selected because, combined, they are the causes of over 50% of the mortality among seniors in the United States, as can be seen in FIGS. 15 to 16 (retrieved from https://vizhub.healthdata.org/gbd-compare/, Institute for Health Metrics and Evaluation, University of Washington). Other example disease conditions are noted below, in Table 1. Such additional diseases/conditions can be included to encompass a majority of diseases/conditions that are relevant for a given target population, or to adequately cover particular individuals within a target population. Residents can be at risk for multiple conditions, and therefore, the Phenotyping Engine also computes an overall mortality risk considering all such factors. Initial risk profiles are uploaded into the data lake where they can be used for further processing by system 100.

Further with respect to FIGS. 15 and 16, health problems can be selected for management by system 100, according to various embodiments, based on prevalence in older persons, and also based upon a potential for reducing adverse effects through monitoring a condition of the resident as well as behaviors of the resident. Because many health problems can be ameliorated through a change in behavior, to varying degrees, to maximize effectiveness of system 100, according to various embodiments, health problems can be selected in order of a probability of reducing adverse effects through monitoring the resident and/or an environment of the resident using system 100. Some examples of such health problems are identified with the encircled letters A-D. While the disclosure focuses on older people, where a greater proportion is at-risk of adverse events, it should be understood that the disclosure can be applied to people of any age in a like manner.

Component 3: Based on the phenotypes assessed, the second AI engine of the system, the Device Matching Engine, automatically selects and/or optimizes a selection of sensors, both wearables and built-in, that need to be assigned to the resident. The AI algorithms developed for this step are based on classification and optimization methodologies. Examples of wearable and built-in sensors, based upon health risk characteristics, are detailed in Table 1, herein. The Device Matching Engine has knowledge of diseases and the measurable/observable characteristics which indicate a state of the disease, and has knowledge of the available sensors and other electronic devices which can be used to gauge or evaluate these characteristics to thereby evaluate changes in risk for individual patients. In this manner, automatic optimization 30) of sensors results in system 100 selecting sensors for individuals without a requirement of input from experts for specific cases. As such, system 100 creates machines in the form of built environments in which individuals can reside, and which includes sensors and other devices which best evaluate relevant aspects of each resident's health over time.

Component 4: Upon assignment to individuals of their respective environments and activation of their sensors or other devices, system 100 continuously collects sensor data which is time-stamped, identified as appropriate, and uploaded into the data lake.

Component 5: The third AI engine, the Predictive Analytics Engine, continuously updates the health risk (probability) predictions based on sensor data and previous predictions, and analyzes trends to detect potential adverse events, and predicts the occurrence of such events over time. Algorithms used in this engine include Bayesian modeling, and detection and forecasting methodologies.

Component 6: The fourth AI engine, the Alerts Analytics Engine, continuously analyzes the output of the Predictive Analytics Engine to trigger alerts and suggest possible interventions, both clinical and environmental, to mitigate the likelihood of potential adverse events. System 100 selectively and automatically sends corresponding alerts to appropriate users, including facility operators, residents, clinical staff, and physicians, who can review the information and decide on appropriate courses of action. Various courses of action can be suggested by system 100 based upon historically successful strategies with similar data scenarios.

Component 7: A user interface with full visualization of all data, including risk profiles, phenotypes, health risk probability, health status, potential adverse events, and alerts, enables a range of users (with corresponding access privileges) to monitor the status of residents over time. The user interface can include graphical data representations of incidents, with severity represented by various colors, where the incidents are overlaid with various time scales. Three examples of graphical data representations that can be displayed in one or more user interfaces are shown in FIGS. 17, 18, and 19. In the example shown in FIG. 17, one or more certain incidents can be represented throughout a 24 hour day clock. Users can select individual incidents to learn more, for example to view other device data at the same period of time, and for a period of time before and after the selected incident. In the example shown in FIG. 18, duration of certain incident(s) can be represented by one or more bar graphs showing incident duration in minutes over historical data, where a normal range of duration can also be indicated. In the example shown in FIG. 19, values of a characteristic of a resident can be shown with historical data graphed over time, such as months, and where a normal range of values of the characteristic can also be indicated. The displayed values also can include a trend line which shows a trend in the values of a characteristic over time. In the example shown in FIG. 19, a displayed trend line indicates a downward trend over time. This information can facilitate diagnosis by health experts and can be used by other experts to improve system 100.

As such, system 100 reduces adverse health effects for at-risk patients. System 100 constructs a unique monitoring environment for each patient, comprising components based upon the health history of the patient, as well as health data for other patients being monitored by system 100, and as available, based further upon health data for the general population, or the largest population for which data is available, and which includes health related data and adverse health effects for people with similar health history to the patients to be monitored by system 100.

When designing the monitoring environment for each patient, system 100 considers the historical health related physical manifestations that preceded adverse health events, for particular sets of health problems.

Such manifestations can include a wide variety of observable characteristics, such as the following limited set of examples: dizziness, unusually frequent trips to the bathroom, changes in food intake, changes in blood pressure/heart rate/respiration, changes in blood glucose, gait instability, fainting, cries for help, changes in body weight, changes in physical activity, wandering, difficulty eating/swallowing, changes in social interaction, changes in habits, changes in hygienic behaviors, changes in sleep patterns, visual changes in the skin, difficulties with vision/hearing/speech/dexterity, and incontinence, as examples. Other measurably characteristics are known to exist. However, these characteristics, at least, can be measured by devices worn upon/within the patient and/or the patient's living environment (residence). Some limited examples of diseases which can give rise to the potential for these observable characteristics are given in Table 1, herein.

System 100 selects devices which can directly or indirectly observe the patient to sense these observable characteristics based upon a probability that the characteristics may arise due to the patient's medical condition, history, and the medical conditions and history of others. The devices can in some cases directly report instances of the observable characteristics, or the devices can trigger another device to electronically report such instances to system 100. According to various embodiments, a report can be made by a device sending an alert message to a selected computer device associated with a medical or health professional. Examples of such devices are given in Table 1, herein, and include as a limited set of examples: blood pressure monitor, heart rate monitor, camera and motion sensing software, respiratory rate monitor, pulse oximeter, transcutaneous CO2 monitor, passive infrared motion sensor, bioimpedance monitor, pulse wave monitor, electrocardiogrameight scale, pillbox, temperature monitor, perspiration monitor, actigraph, brachial index monitor, acoustic sensor, brainwave monitor, geofence, nutrition software, self-assessment software, motion detecting sleep mat, door contact on doorway, sensor on toilet, time tracking sensor, retina scanner, urine analyzer, blood glucose monitor, transcutaneous glucose monitor, liquid dispensing monitor, air quality sensor, light sensor, balance board, telephone, and/or computer.

System 100 can use any known algorithm for selecting the devices to position. However, in an embodiment of the disclosure, artificial intelligence algorithms are used, for which a direct pathway between consideration of medical condition and history and the selection of devices cannot be defined with specificity. Further, system 100 can use past results to continue to improve the selection of devices, where improvement includes early detection of the relevant health manifestations/characteristics which enables early health related intervention. Other improvements can include the selection of lower cost, more readily obtainable, lighter, and smaller monitoring devices, as examples.

After system 100 specifies the devices to be positioned, the devices are positioned by humans or machines with instructions from system 100. At that point, system 100 begins monitoring the patient using the devices. When the devices indicate manifestation of characteristics which correlate with an existing or potential health problem or adverse event, system 100 next determines if intervention is required. Again, system 100 can use artificial intelligence which improves over time based upon results which result in fewer health problems and adverse events. More particularly, system 100 uses the device data to determine whether the patient is at a predetermined higher probability of an adverse health event relative to a baseline probability for the patient. The baseline is derived from the patient's medical history as well as prior observations of the patient, and others with similar health problems, by system 100. In an embodiment, the patient or health practitioner can indicate to system 100 that a particular manifestation has particular risk level, e.g. low, medium, or high, of an adverse event, and system 100 can re-weight such manifestations accordingly in the future. Detection of manifestations are added to the historical database accessible to system 100, as part of an automated process of continuous improvement.

Thus, system 100 includes continuous, technology-driven, health and sensor data collection, enabling a unique and scalable model for data capture, storage, artificial intelligence and predictive analytics processing, that transforms a residential environment into a “Living Laboratory” for preventing adverse events. Captured data streams are interpreted and translated by the expert system yielding preventive health interventions and therapeutic behavioral/environmental modifications to maximize health-related quality of life and longevity of residents.

Table 1 details data that forms part of the analytic engines of system 100 described above, according to various embodiments, for 11 illustrative diseases, although any number of diseases can be addressed by system 100. The data forming part of the analysis as shown in Table 1 includes health history considered in view of a presentation of symptoms, any gender related factors, and severe adverse outcomes to mitigate. Other factors can be considered by system 100, as described herein. Next, system 100 selects a set of sensor/diagnosis tools which have features selected to target and address the foregoing health factors. Certain tools are provided in the form of devices placed within the “built environment” or residence, and other devices are carried, used, worn by, or implanted within the resident. Data from the tools are continuously uploaded to the data lake for analysis as described herein. The data thus provided enables system 100, according to various embodiments, to provide real time or near real time advance warning of a potential worsening of a patient's condition which may give rise to an adverse outcome.

TABLE 1

| Part 1 of 6 | Cardiovascular Disease | Hyperlipidemia |
|---|---|---|
| Presentation of | ↑ Chest pain, ↑ shortness of breath, | BP and HR changes |
| Symptoms | changes in sleeping pattern (PND), | |
| | changes in activity, pain in | |
| | neck/jaw/throat/upper abdomen, | |
| | extremity numbness | |
| Gender | Roughly gender equivalent: AHA, 2019. | No gender differences observed from |
| (Differences/Common | Adults 8Q+:F (89.3%) v M | 75-90 yo |
| Occurrence) | (91.8%). 86% of people >80 yo with heart | |
| | disease | |
| Most Severe Adverse | MI, congestive heart failure, CAD, | Advanced cardiovascular disease: |
| Outcome related | cardiac arrest, cardiac arrhythmia | stroke, MI |
| to Comorbidity | | |
| Sensor/Diagnosis | Biometric analysis of BP, HR (patch or | Minimal non-invasive techniques, |
| Tool | wearable device). Gait instability, detect | Visual and IR guided sensors for |
| | fainting prior to occurrence | monitoring cholesterol and glucose, |
| | (environmental sensor). Respiratory' rate | through skin conductivity tests |
| | with pulse oximeter (wearable). | |
| | Transcutaneous C02 monitoring- | |
| | combine both respiratory' and cardiac | |
| | analysis | |
| Built Environment | Gate instability, fall detection Passive | Visual and IR guided sensors PIR |
| | infrared motion sensors Digital bio- | motion sensors |
| | impedance scale (pulse composition, | |
| | pulse wave velocity, and ambient C02) | |
| Wearable Device | Biometric analysis (pulse wave velocity, | Biometric analysis of BP, HR, temp., |
| | pulse), Transcutaneous CG2 -ECG | Perspiration monitor, O2 saturation, |
| | through wearable (auto-triggered, HR | ECG through wearable (continuous |
| | triggered, pt triggered) Example: Rhythm | tracking and reading for accurate, |
| | Express RK-1 -Afib (24/7 tracking) | predictive outcomes) |

| Part 2 of 6 | Hypertension | Peripheral Vascular Disease (FVD) |
|---|---|---|
| Presentation of | ↑.HR, ↑ BP, dizziness, perspiration, trips | ↓ Activity/mobility, claudication, edema |
| Symptoms | to bathroom (prescription of diuretic), | of lower extremity, skin changes |
| | kidney issues. | |
| Gender | After 75 yo = Women > Men, Pre- | 80+: equivalent rates for M and F, Risk |
| (Differences/Common | hypertensive: Men > Women. Women | factors for women- diabetes, smoking, |
| Occurrence) | show protection from age 20-44 | obesity |
| Most Severe Adverse | Stroke, advanced cardiovascular | Atherosclerotic disease leading to limb |
| Outcome related | disease (MI, CHF), renal failure | ischemia and possible amputation, |
| to Comorbidity | | immobility, risk of pulmonary embolism |
| | | or DVT |
| Sensor/Diagnosis | Biometric analysis of BP, HR (wearable | Assess changes in sitting, standing, |
| Tool | sensor), compare throughout day. | walking (wearable) Changes in HR |
| | Perspiration monitoring through | Assessing ankle brachial index (ABI) |
| | wearable | through wearable biometric patch on |
| | | LE for high risk individual |
| Built Environment | Digital bioimpedance scale (pulse | PER motion sensor's geofence |
| | composition, pulse wave velocity, and | (movement pattern) |
| | ambient CO2) Digital balance board for | |
| | balance testing, Electronic pillbox - med | |
| | adherence | |
| Wearable Device | Biometric analysis of BP, HR, temp. | Wearable actigraph (e.g. a worn |
| | Perspiration monitor, O2 sat. ECG | accelerometer): sitting, standing, |
| | through wearable (continuous tracking | walking ankle, brachial index (ABI): |
| | and reading for accurate, predictive | wearable patch |
| | outcomes) | |
| Part 3 of 6 | Dementia/Cognitive Decline | Depression |
| Presentation of | Wandering, memory loss, difficulty | ↓ Social interaction, ↑time spent in |
| Symptoms | eating and walking | room, ↓ eating, alteration in sleep cycle |
| | | (↑/↓ sleeping), ↓ hygiene, ↓ time |
| | | bathing |
| Gender | Women > Men, 2/3 of those diagnosed | Depression and depression related |
| (Differences/Common | with AD are women, Women more likely | symptoms: Women > Men |
| Occurrence) | to have dementia (2:1) | |
| Most Severe Adverse | Falls (orthopedic injury), malnutrition, | Development of psychosocial |
| Outcome related | migraine, decreased quality of life | disorders, decrease in quality of life, |
| to Comorbidity | | risk of suicide, ↑ risk of Alzheimer's |
| Sensor/Diagnosis | Daily problem solving questions | Changes in activity/movement around |
| Tool | (neurological exam simulation) through | facility (environmental sensor), Activity |
| | watch, assess correctness of responses | level, geo-fence tracking (enviro + |
| | and time to respond (on wearable), | wearable), intervention for highest |
| | Geofence tracking (environment | risks could ping residents to meet/send |
| | sensor), Nutrition assessment | to high social areas. Residents send |
| | | notification to provider about other |
| | | residents |
| Built Environment | Cognitive self-assessment w/computing | .PIR motion sensors/geofence (room |
| | device, PC & Telephone use, duration . | transitions) .Socialization (est. |
| | Geofence or PIR sensors; socialization, | loneliness) . Sleep mats: quality, |
| | speed, pattern, Sleep mats: quality, | duration, sleep stages .Cognitive self- |
| | duration, sleep stages, Electronic pill | assessment w/computing device (or |
| | box: med. tracking. Door contact | tablet) |
| | sensors - (refrigerator for nutrition) | |
| Wearable Device | Wearable actigraph/physiological | . Biometric analysis/actigraph . HR, |
| | metrics, Socialization, behavior. Nutrition | resp. rate, while sleep or throughout |
| | tracking (location and patterns of | day; posture. . Cognitive self- |
| | movement) | assessment w/wearable device |
| | | (collected weekly) |

| Part 4 of 6 | Diabetes (Type 2) | Falls/Immobility |
|---|---|---|
| Presentation of | ↑ time in bathroom/urination, ↑ | Dizziness, instability, gate alteration, ↓ |
| Symptoms | dehydration, changes in diet, ↑ thirst, | activity |
| | visual changes, tingling/numbness in | |
| | extremities, ↑ skin infection | |
| Gender | DM II rates similar for M and F in late- | Non-fatal falls: Women > Men; women |
| (Differences/Common | life. Higher for men in mid-life | 50% greater risk |
| Occurrence) | | |
| Most Severe Adverse | Retinopathy, PVD, Coronary artery | Orthopedic injury, exacerbation of |
| Outcome related | disease (CAD), renal failure, urinary | comorbidities, neurologic injury, risk of |
| to Comorbidity | incontinence | infection, hemorrhagic |
| Sensor/Diagnosis | Bathroom frequency, dietary changes, | Gate sensors in floor of facility. Activity |
| Tool | weight change (sensors in ground | monitoring/disorganized movement. |
| | detecting movement and weight | Periodic neurologic exam assessed |
| | distribution). Transcutaneous glucose | through wearable device. Acceleration |
| | monitoring system integrated into | and motion trajectory assessment in |
| | CP/wearable platform + integration of | wearable. Alert button on wearable |
| | additional biometric factors | |
| Built Environment | Movement or PIR sensors (movement | Gate instability, fall det. Passive |
| | patterns), Door contact sensors | Infrared motion sensors Digital balance |
| | (bathroom and kitchen/fridge). Toilet | board for balance testing Computing |
| | sensors (in toilet, flush mechanism) | device- cognitive activities/self- |
| | | assessments for physical therapy (PT) |
| Wearable Device | Biometric analysis of BP, HR, A1C . | Biometric analysis (physical activity, |
| | Transcutaneous glucose monitoring | sleep). .Predictive, algorithm integrated |
| | system, Diet changes (water | into wearable and enviro. |
| | consumption) | |

| | Chronic lower respiratory disease | |
|---|---|---|
| Part 5 of 6 | (COPD, emphysema) | Urinary incontinence |
| Presentation of | Changes in HR and respiratory rate, | ↑ time in bathroom, ↓ sleeping, ↑ |
| Symptoms | changes in activity, changes in | activity, ↑ dehydration |
| | sleep/wake cycle, body position (↓ time | |
| | in supine position) | |
| Gender | COPD: Women > Men | No gender discrimination among UI |
| (Differences/Common | | overall. Women experience stress |
| Occurrence) | | incontinence more than functional |
| | | incontinence |
| Most Severe Adverse | Respiratory failure, pneumonia, risk of | Renal insufficiency, development of |
| Outcome related | infection, (chronic lower respiratory | cancer (bladder, kidney), Prostate |
| to Comorbidity | disease 3rd leading cause of death of | disease (men), sign of Parkinson's |
| | adults over 65 yo) | disease or Alzheimer's disease |
| Sensor/Diagnosis | Patterns in movement, stopping-starting, | Frequency/patterns in trips to |
| Tool | time for consistent movement Body | bathroom. (Prostate disease in men). |
| | position analysis with environmental | Time sleeping/in bed. Toilet sensor |
| | sensor (more time supine/upright). | that measures # of flushes and/or |
| | Transcutaneous CO2 monitoring and | volume of urine output |
| | pulse oximetry. UV filtration, air quality | |
| | assessment to improve outcomes for at | |
| | risk resident | |
| Built Environment | PIR sensors & body position analysis | Movement or PIR sensors (sleep |
| | Sleep mats: quality, duration, sleep | patterns, bathroom) Door contact |
| | stages UV filtration, air quality | sensors (bathroom and kitchen/fridge). |
| | assessment to improve outcomes for at | Toilet sensors (in toilet, flush |
| | risk residents | mechanism) Sleep |
| Wearable Device | Transcutaneous CO2 monitoring and | Actigraph: sleep, trips to bathroom w/ |
| | pulse oximetry. HR, pulse, pulse wave | wearable Physiological analysis- skin |
| | velocity, O2 saturation | hydration, indicators of dehydration. |
| | | Electrical stimulation (similar to IB |
| | | Stim) |

| Part 6 of 6 | Visual impairments |
|---|---|
| Presentation of | ↑ Eye pain, blurred vision (resident |
| Symptoms | determined), floating objects, alteration |
| | in activity patterns, light sensitivity: ↑time |
| | in dark, ↓ time outside |
| Gender | No gender differences observed; greater |
| (Differences/Common | risk associated with age. HRQOL lower |
| Occurrence) | for women with visual impairments |
| Most Severe Adverse | Glaucoma, retinal detachment, macular |
| Outcome related | degeneration, risk of falling, risk of |
| to Comorbidity | depression/↓ social interaction, risk of |
| | cognitive decline |
| Sensor/Diagnosis | Room sensors: changes in lighting: |
| Tool | ↑time in dark, ↓ time outside. Dizziness + |
| | visual ping when experiencing |
| | symptoms, located in room to notify |
| | provider |
| Built Environment | Room sensors tracking lighting (on/off, |
| | duration, time). Digital balance board for |
| | balance testing. Motion sensor - |
| | dizziness, trigger HC provider if off |
| | pattern. PC & Telephone use, duration |
| Wearable Device | Motion tracking. Sleep behavior - HR, |
| | sleep mat behavior |

Further, the disclosure enables a translation of health data collected from sensor technology and/or wearable devices into actionable health or behavioral interventions. AI can be used to detect/interpret abnormalities and communicate these in real time (sufficiently timely to prevent an adverse health event) with local health care providers. Agreements can be established with local health care providers to proactively engage with systems of the disclosure to implement a prospective care model which continuously improves.

Data from the sensors/diagnostic tools can be transferred to the data lake via the internet. Certain devices may communicate directly, such as IoT enabled devices, and other devices may communicate information over a local wireless or wired network, whereupon a designated device can collect and forward the data for devices which are not capable of connecting directly. Still further, certain devices may be observed by another device, or the devices may signal another device through a wired or wireless connection, and the other device may then communicate information related to the device to system 100.

All references cited herein are expressly incorporated by reference in their entirety. There are many different features of the present disclosure and it is contemplated that these features may be used together or separately. Unless mention was made above to the contrary, it should be noted that all of the accompanying drawings are not to scale. Thus, the disclosure should not be limited to any particular combination of features or to a particular application of the disclosure.

Further, it should be understood that variations and modifications within scope of the disclosure might occur to those skilled in the art to which the disclosure pertains. Accordingly, all expedient modifications readily attainable by one versed in the art from the disclosure set forth herein that are within the scope of the present disclosure are to be included as further embodiments of the present disclosure.

What is claimed is:

1. A computer-implemented system for reducing adverse health effects for at-risk patients, comprising:
one or more computing devices executing instructions, stored on media readable by the one or more computing devices, to:
store data pertaining to historical physical manifestations of various health problems in a set of at-risk people;
store data pertaining to at-risk patients including health problems of each patient:
for each patient, select a plurality of electronic monitoring devices which each detect at least one of the physical manifestations of a health problem of the patient and report the selection of devices whereby the devices can be positioned upon at least one of the patient or an environment in which the patient lives;
for each patient, based on the electronic monitoring devices that have been positioned, electronically monitor the devices to determine if one or more manifestations of a health problem of the patient has taken place;
use the determination to evaluate whether the patient is at a predetermined higher probability of an adverse health effect relative to a baseline probability for the patient, and report, by sending an alert message to a selected computer device associated with a medical or health professional, the higher probability whereby assistance can be directed to the patient; and
add data pertaining to the predetermined higher probability to the data pertaining to historical physical manifestations.

2. The system of claim 1, wherein the electronic monitoring devices are selected from the group of:
blood pressure monitor, heart rate monitor, camera and motion sensing software, respiratory rate monitor, pulse oximeter, transcutaneous CO2 monitor, passive infrared motion sensor, bioimpedance monitor, pulse wave monitor, electrocardiograph, weight scale, pillbox, temperature monitor, perspiration monitor, actigraph/wearable accelerometer, brachial index monitor, acoustic sensor, brainwave monitor, geofence, nutrition software, self-assessment software, motion detecting sleep mat, door contact on doorway, sensor on toilet, time tracking sensor, retina scanner, urine analyzer, blood glucose monitor, transcutaneous glucose monitor, liquid dispensing monitor, air quality sensor, light sensor, balance board, telephone, or computer, or any combination thereof.

3. The system of claim 1, wherein at least one of the electronic monitoring devices is a biometric monitoring device.

4. The system of claim 1, wherein at least one of the electronic monitoring devices is a device positioned upon the patient inside the body of the patient.

5. The system of claim 1, wherein the electronic monitoring devices positioned upon the environment include at least one of a motion sensor, weight scale, acoustic sensor and/or geofence.

6. The system of claim 1, wherein the electronic monitoring devices positioned upon the environment include a device for monitoring a position of the patient within the environment.

7. The system of claim 1, wherein the electronic monitoring devices include devices which have IoT (Internet of Things) capabilities enabling independent communication of data over the Internet.

8. A computer-implemented method for reducing adverse health events for at-risk patients, comprising:
collecting, with a processor accessing records in a computer database, objective health data of at least one individual resident patient (resident) using a health history of the resident, which is stored in one or more records in the computer database, and objective health data collected from a current evaluation of cognitive abilities, functional abilities, and comorbidities, of the resident;
using the collected objective health data to assign (a) independent living to a resident with normal cognitive function and no functional disabilities, (b) assisted living to a resident unable to carry out a plurality of activities of daily living, and (c) memory care living to a resident with a dementia diagnosis;
if the collected objective health data of a resident are used to assign (b) or (c), provide at least one of wearable sensors on the resident and sensors within the living environment of the resident, which provide sensor data pertaining to a real time health state of the resident living within the assisted living or memory care environment, the type of sensor selected being based upon the collected objective health data;
communicate the sensor data in real time to at least one of a health expert and a computer algorithm to analyze the sensor data to determine if the resident requires assistance; and
reevaluate a correlation of the collected objective health data, selection of sensors, and analysis of sensor data for a plurality of patients to reduce a requirement of assistance of patients.
